(19) United States
(12) Patent Application Publication
(10) Pub. No.: US 2014/0170193 A1
(43) Pub. Date: Jun. 19, 2014

(54) Shaving Foam Wipe Unit

(71) Applicant: Gunnar Myhre, Drammen (NO)

(72) Inventor: Gunnar Myhre, Drammen (NO)

(21) Appl. No.: 13/961,033

(22) Filed: Aug. 7, 2013

(30) Foreign Application Priority Data
Dec. 18, 2012 (TW) 101224530

(51) Int. Cl. A61K 8/02; A61K 8/34; A61K 8/46; A61K 8/36; A61Q 9/02; A61K 8/44

(52) U.S. Cl. CPC A61K 8/0208; A61Q 9/02; A61K 8/442; A61K 8/463; A61K 8/361; A61K 8/34
USPC 424/401; 424/73

(57) ABSTRACT

A shaving foam wipe unit includes a package unit and at least one emollient foam wipe received in the package unit. The emollient foam wipe includes a lamellar carrier and a skin moisturizer. The skin moisturizer is disposed on a surface of the lamellar carrier or impregnated in the lamellar carrier. When the skin moisturizer of the emollient foam wipe is applied on a user's facial skin, a moisturizing layer is produced on the skin surface for producing foam. Thereby, not only that the emollient foam wipe can provide good moisturizing effect for shaving, convenience of usage, good portability and friendly to the environment are also the advantages.

### BACKGROUND OF THE INVENTION

The present invention relates to a shaving foam wipe unit and, more particularly, to an emollient foam wipe for producing a moisturizing layer on the skin to be shaved.

Most men use shaving cream or foam for moisturizing the skin during beard or mustache shaving. Specifically, in order to avoid the skin being harmed by sharp razors, shaving foam has to be applied on the skin to be shaved first. Thereby, a moisturizing layer can be produced on the skin surface to moisture and lubricate facial hairs speedily. Then, a razor is used to shave off the facial hairs. Conventional shaving foam is filled in a pressurized bottle in a liquid form. A button is disposed on the pressurized bottle for a user to press in order to spray the foam from the pressurized bottle, and the sprayed foam is spread on the skin by the user.

Although conventional bottled shaving foam or cream can provide certain effectiveness, the pressurized bottle filled with shaving foam has to be stayed away from high temperatures and high pressures during the storage life. Thus, it has limitations in usage. Additionally, the pressurized bottle is also inconvenient for carrying around and, thus, has poor portability.

Therefore, an emollient foam wipe for shaving is required for improving the above mentioned problems and drawbacks.

### BRIEF SUMMARY OF THE INVENTION

It is an objective of the present invention to provide a shaving foam wipe unit which includes at least one emollient foam wipe for applying on a user's facial skin to be shaved. Thereby, a moisturizing layer can be produced on the skin surface for producing a rich and fine lathering to moisture the facial hairs speedily and facilitate shaving.

To achieve this and other objectives, a shaving foam wipe unit of the present invention includes at least one emollient foam wipe having a lamellar carrier and a skin moisturizer. The lamellar carrier is made of an absorbent material and includes a first surface for applying on a facial skin of a user. The skin moisturizer is disposed on the first surface of the lamellar carrier or impregnated in the lamellar carrier. A moisturizing layer is produced on a surface of the facial skin for producing foam when the skin moisturizer of the emollient foam wipe is applied on the facial skin of the user.

In a preferred form, the shaving foam wipe unit further includes a package unit having an internal accommodation chamber therein, and the at least one emollient foam wipe is received in the accommodation chamber of the package unit in a foldable form or in a roll form.

In a preferred form, the skin moisturizer is made of a mixture including water, sodium lauroyl glutamate for interfacial activity, cocamidopropyl betaine or sodium lauryl sulfate for interfacial activity, myristic acid for moisturizing, menthol, and dexpanthenol for softening beards. The lamellar carrier further includes a second surface opposed to the first surface, with the first and second surfaces of the lamellar carrier containing the skin moisturizer.

The present invention will become clearer in light of the following detailed description of illustrative embodiments of this invention described in connection with the drawings.

### DESCRIPTION OF THE DRAWINGS

FIG. 1 shows a schematic view of a shaving foam wipe unit according to a first embodiment of the present invention.

FIG. 2 shows a perspective view of an unfolded emollient foam wipe in FIG. 1.

FIG. 3 shows a schematic view of a shaving foam wipe unit according to a second embodiment of the present invention.

FIG. 4 shows a schematic view of a shaving foam wipe unit according to a third embodiment of the present invention.

FIG. 5 is a schematic view similar to FIG. 4, with a cover of a package unit in FIG. 4 opened.

FIG. 6 shows a schematic view of a shaving foam wipe unit according to a fourth embodiment of the present invention.

### DETAILED DESCRIPTION OF THE INVENTION

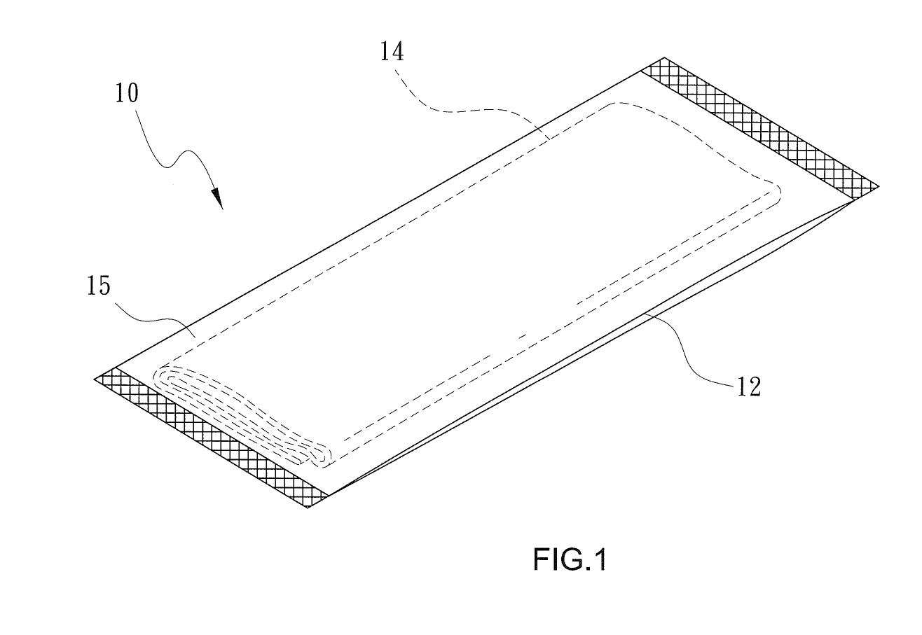

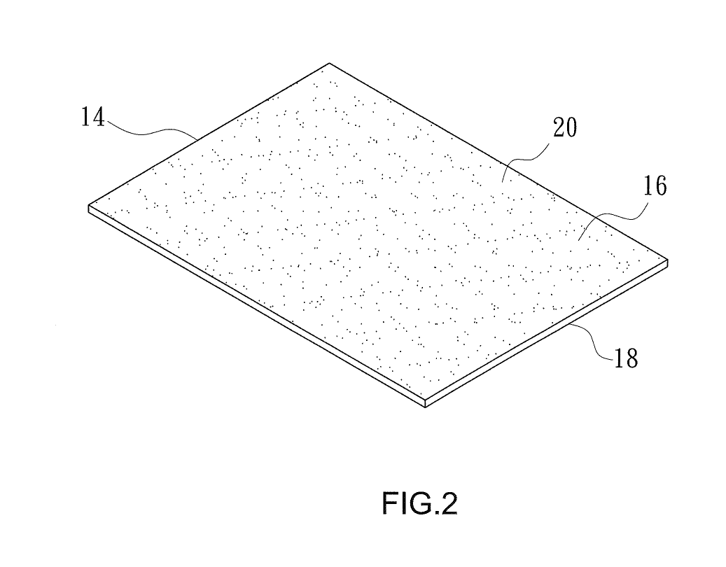

A shaving foam wipe unit 10 according to a first embodiment of the present invention is shown in FIGS. 1 and 2 of the drawings and includes a package unit 12 and an emollient foam wipe 14 received in the package unit 12. In this embodiment, the package unit 12 is a packaging bag with an internal accommodation chamber 15.

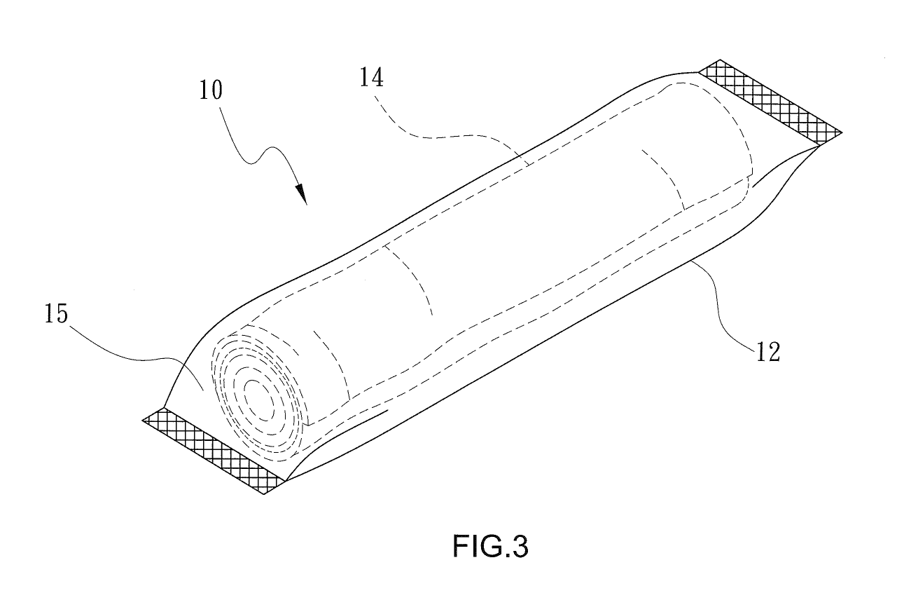

The emollient foam wipe 14 includes a lamellar carrier (not labeled). The lamellar carrier is made of a soft and absorbent material so that the emollient foam wipe 14 can be received in the accommodation chamber 15 of the package unit 12 in a foldable form as shown in FIG. 1; or, can be received in the accommodation chamber 15 of the package unit 12 in a roll form as shown in FIG. 3. The lamellar carrier can be selected from at least one of paper towel, cotton cloth, fiber paper, non-woven fabric, spunlace non-woven fabric and hydro knit fabric. The lamellar carrier includes a first surface 16 for applying on a user's facial skin and a second surface 18 opposed to the first surface 16.

The emollient foam wipe 14 further includes a skin moisturizer 20. The skin moisturizer 20 is disposed on the first surface 16 of the carrier by spreading or spraying, or impregnated in the carrier so that the first and second surfaces 16 and 18 of the carrier contain the skin moisturizer 20. The components of the skin moisturizer 20 include water, sodium lauroyl glutamate for interfacial activity, cocamidopropyl betaine and/or sodium lauryl sulfate (SLS) for interfacial activity and cleaning and foaming, myristic acid for moisturizing, menthol for cooling and refreshing, dexpanthenol for softening beard or mustache hairs, and fragrances.

When the shaving foam wipe unit 10 is embodied, the emollient foam wipe 14 is taken out from the package unit 12, and the first surface 16 of the emollient foam wipe 14 is applied and moved on the facial skin to be shaved (the second surface 18 can also be applied on the facial skin to be shaved if the second surface 18 is provided with the skin moisturizer 20). Thereby, a moisturizing layer can be produced on the facial skin surface to be shaved for producing a rich and fine lathering to moisture the facial hairs speedily. Then, a razor can be used to shave off the beards or facial hairs easily and smoothly.

Besides the moisturizing and lubricating effects provided by the emollient foam wipe 14 of the shaving foam wipe unit 10 of the present invention for facilitating shaving, the emollient foam wipe 14 received in the package unit 12 is also convenient for using and carrying around, as well as environmentally friendly. Thus, the expected objectives and effects of the present invention are achieved.

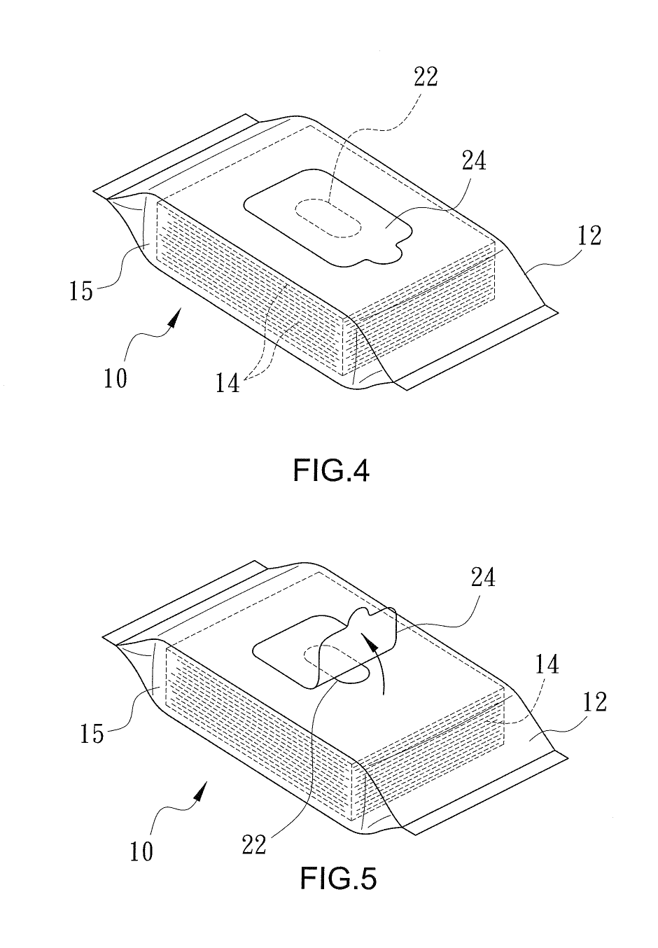

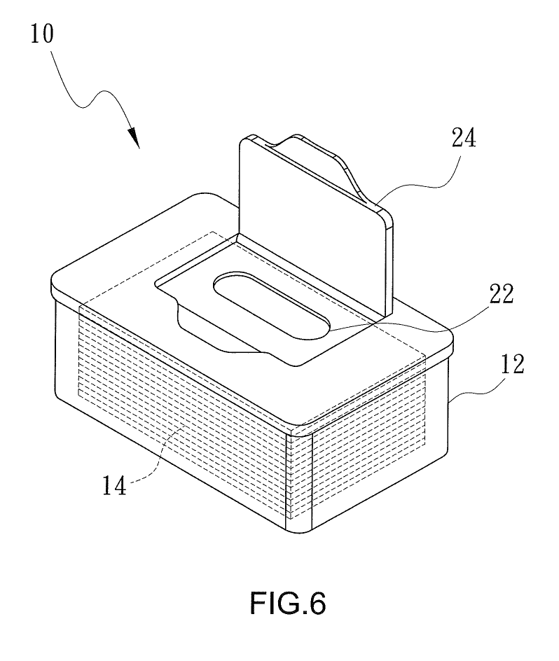

FIGS. 4 and 5 show a shaving foam wipe unit 10 according to a third embodiment of the present invention and including a package unit 12 and a plurality of the emollient foam wipes 14 received in the package unit 12. The package unit 12 has an internal accommodation chamber 15 for receiving the emollient foam wipes 14. An opening 22 is provided in an upper portion thereof and sealed by a cover 24. FIG. 6 shows a shaving foam wipe unit according to a fourth embodiment of the present invention and including a package unit 12 and a plurality of the emollient foam wipes 14 received in the package unit 12. In this embodiment, the package unit 12 is embodied as a box and includes an opening 22 sealed by a cover 24 disposed on an upper portion of the package unit 12. When one of the emollient foam wipes 14 needs to be taken out, the cover 24 is simply opened. Therefore, it is very convenient for using.

Thus since the invention disclosed herein may be embodied in other specific forms without departing from the spirit or general characteristics thereof, some of which forms have been indicated, the embodiments described herein are to be considered in all respects illustrative and not restrictive. The scope of the invention is to be indicated by the appended claims, rather than by the foregoing description, and all changes which come within the meaning and range of equivalency of the claims are intended to be embraced therein.

1. A shaving foam wipe unit comprising:
at least one emollient foam wipe including a lamellar carrier and a skin moisturizer, with the lamellar carrier made of an absorbent material and including a first surface for applying on a facial skin of a user, with the skin moisturizer disposed on the first surface of the lamellar carrier or impregnated in the lamellar carrier, with a moisturizing layer produced on a surface of the facial skin for producing foam when the skin moisturizer of the emollient foam wipe is applied on the facial skin of the user.

2. The shaving foam wipe unit according to claim 1, wherein the skin moisturizer is made of a mixture including water, sodium lauroyl glutamate for interfacial activity, cocamidopropyl betaine or sodium lauryl sulfate for interfacial activity, myristic acid for moisturizing, menthol, and dexpanthenol for softening beards.

3. The shaving foam wipe unit according to claim 2, wherein the lamellar carrier further includes a second surface opposed to the first surface, with the first and second surfaces of the lamellar carrier containing the skin moisturizer.

4. A shaving foam wipe unit comprising:
a package unit having an internal accommodation chamber therein; and
an emollient foam wipe received in the accommodation chamber of the package unit and including a lamellar carrier and a skin moisturizer, with the lamellar carrier made of an absorbent material and including a first surface for applying on a facial skin of a user, with the skin moisturizer disposed on the first surface of the lamellar carrier or impregnated in the lamellar carrier, with a moisturizing layer produced on a surface of the facial skin for producing foam when the skin moisturizer of each emollient foam wipe is applied on the facial skin of the user.

5. The shaving foam wipe unit according to claim 4, wherein the skin moisturizer is made of a mixture including water, sodium lauroyl glutamate for interfacial activity, cocamidopropyl betaine or sodium lauryl sulfate for interfacial activity, myristic acid for moisturizing, menthol, and dexpanthenol for softening beards.

6. The shaving foam wipe unit according to claim 5, wherein the lamellar carrier further includes a second surface opposed to the first surface, with the first and second surfaces of the lamellar carrier containing the skin moisturizer, with the emollient foam wipe received in the package unit in a foldable form or in a roll form.

7. A shaving foam wipe unit comprising:
a package unit having an internal accommodation chamber therein; and
a plurality of emollient foam wipes received in the accommodation chamber of the package unit, with each emollient foam wipe including a lamellar carrier and a skin moisturizer, with the lamellar carrier made of an absorbent material and including a first surface for applying on a facial skin of a user, with the skin moisturizer disposed on the first surface of the lamellar carrier or impregnated in the lamellar carrier, with a moisturizing layer produced on a surface of the facial skin for producing foam when the skin moisturizer of each emollient foam wipe is applied on the facial skin of the user.

8. The shaving foam wipe unit according to claim 7, wherein the skin moisturizer is made of a mixture including water, sodium lauroyl glutamate for interfacial activity, cocamidopropyl betaine or sodium lauryl sulfate for interfacial activity, myristic acid for moisturizing, menthol, and dexpanthenol for softening beards.

9. The shaving foam wipe unit according to claim 8, wherein the lamellar carrier further includes a second surface opposed to the first surface, with the first and second surfaces of the lamellar carrier containing the skin moisturizer.

10. The shaving foam wipe unit according to claim 8, wherein each of the emollient foam wipes is received in the package unit in a foldable form or in a roll form.
